(19) United States
(12) Patent Application Publication
(10) Pub. No.: US 2026/0216712 A1
(43) Pub. Date: Jul. 30, 2026

(54) CATALYST FOR CONVERTING REFORMATE TO XYLENES

(71) Applicant: KING FAHD UNIVERSITY OF PETROLEUM AND MINERALS, Dhahran (SA)

(72) Inventors: Ziyauddin S. QURESHI, Dhahran (SA); Syed A. ALI, Dhahran (SA)

(73) Assignee: KING FAHD UNIVERSITY OF PETROLEUM AND MINERALS, Dhahran (SA)

(21) Appl. No.: 19/039,344

(22) Filed: Jan. 28, 2025

(51) Int. Cl. B01J 29/76; B01J 21/08; B01J 35/30; B01J 35/61; B01J 35/63; B01J 37/00; B01J 37/02; B01J 37/03; B01J 37/06; B01J 37/08; C07C 6/06

(52) U.S. Cl. CPC B01J 29/7615; B01J 21/08; B01J 35/397; B01J 35/617; B01J 35/633; B01J 35/635; B01J 37/0018; B01J 37/009; B01J 37/0201; B01J 37/036; B01J 37/06; B01J 37/08; C07C 6/06; B01J 2229/20

(57) ABSTRACT

A catalyst composition and a method for the transalkylation of heavy aromatics includes core-shell mesoporous silica-beta zeolite (BZ@MSi) composite particles including a mesoporous silica (MSi) shell, a beta zeolite (BZ) core particle, and nickel (Ni). The mesoporous silica shell encompasses the BZ core particle, with a thickness ranging from 10 nm to 100 nm. The thickness of the mesoporous silica shell is ranges from 10 nm to 100 nm. Further, Ni is dispersed over the BZ@MSi composite particles in an amount ranging from 0.1 wt. % to 10 wt. % relative to the total weight of the BZ@MSi composite particles.

### STATEMENT REGARDING PRIOR DISCLOSURE BY THE INVENTORS

Aspects of the present disclosure are described in Ali, S., et al., “Influence of feed contamination on the conversion of heavy reformate and toluene over a composite hierarchical zeolite catalyst” published in Issue 9, Reaction Chemistry and Engineering on Feb. 13, 2024, which is incorporated herein by reference in its entirety.

### BACKGROUND

### Technical Field

The present disclosure is directed towards a catalyst composition and, more particularly, relates to mesoporous silica beta zeolite catalysts for the conversion of heavy reformate to xylenes.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Xylenes (C₆H₄(CH₃)₂) are aromatic hydrocarbons used in the production of plastics, synthetic fibres, and solvents. Xylenes are traditionally produced via catalytic reforming and transalkylation of heavy aromatics. However, conventional methods often face challenges such as low selectivity, poor yield, and catalyst deactivation under energy-intensive conditions. The transalkylation process offers a pathway to convert heavy reformate and toluene into valuable xylenes, meeting both economic and industrial demands (See: Ali, S., et al., Parametric study of dealkylation-transalkylation reactions over mordenite-based bi-functional catalysts, Applied Catalysis A: General, Volume 393, Issues 1-2, 2011, pages 96-108).

Xylenes may be efficiently produced from toluene and aromatic hydrocarbons through transalkylation, a catalytic process that redistributes alkyl groups. Among the xylene isomers, (ortho-, meta-, and para-xylene), para-xylene is in higher demand due to its role in producing polyethylene terephthalate (PET) for packaging applications [See: Ashraf, M. et al., Ind. Eng. Chem. Res. 2013, 52, 38, 13730-13737]. P-xylene is also used in manufacturing terephthalic acid and dimethyl terephthalate, used in for polyester production. The annual demand for p-xylene is growing at a rate of 6% to 8%, driven by the widespread use of p-xylene in various downstream processes [See: Precedence Research, Xylene Market Size, Share, and Trends 2024 to 2033]. The growing demand of xylenes highlights the importance of optimizing production methods like transalkylation, in order to meet industrial requirements efficiently.

In general, transalkylation is a petrochemical process that transfers alkyl groups between aromatic hydrocarbons to efficiently produce valuable compounds like xylenes and benzene. In refineries, the process converts heavy reformate, rich in trimethyl benzenes (TMBs) and methyl ethyl benzenes (MEBs), into xylenes. However, the transalkylation process faces drawbacks such as, but not limited to, catalyst deactivation caused by coking, requiring frequent regeneration, and limited selectivity for desired xylene isomers. Additionally, high operating temperatures and pressures increase energy consumption and operational costs, highlighting the requirement for improved catalyst design and process optimization. Catalysts with strong acid sites and high mesoporosity may enhance the selectivity for p-xylene. The optimization minimizes by-products, improving efficiency [S. H. Cha and co-researchers, 1,2,4-Trimethylbenzene disproportionation over large-pore zeolites: An experimental and theoretical study].

C₉ aromatic hydrocarbons such as TMBs and MEBs, present in heavy reformate, may be efficiently converted into xylenes using solid acid catalysts. In addition, several research articles focus on C₉ aromatics (1,2,4-TMB, 1,2,3-TMB, and 1,3,5-TMB), including their model compounds and mixtures of TMBs and MEBs, used as model feeds for transalkylation with toluene over various zeolites [See: Das, J., et al., Transalkylation and disproportionation of toluene and C9 aromatics over zeolite beta, Catal Lett 23, 161-168, 1994]. The commercially applied transalkylation processes include ARCO's Xylene-Plus, UOP's Tatoray, Mobil Oil's MTP, Exxon Mobil's Transplus, SK's ATA and Sinopec's S-TDT [See: Cha, S., et al., 1,2,4-Trimethylbenzene disproportionation over large-pore zeolites: An experimental and theoretical study, Journal of Catalysis, Volume 323, 2015, Pages 145-157].

Zeolites, with the varying acidity and pore structure, are used heavily in upgrading heavy reformate to xylenes through transalkylation [See: Serra, J., et al., Optimizing the conversion of heavy reformate streams into xylenes with zeolite catalysts by using knowledge base high-throughput experimentation techniques, Journal of Catalysis, Volume 232, Issue 2, 2005, Pages 342-354]. The catalytic activity in transalkylation is highly influenced by the pore size and acidity, with larger pore zeolites enhancing xylene formation. Lewis's acid sites promote toluene/TMB disproportionation, while Brønsted acid sites favor TMB isomerization [See: Almulla, F., et al., Ind. Eng. Chem. Res., 2017, 56, 35, 9799-9808]. Several medium pore zeolites (ZSM-5, IM-5, NU-87, and MCM-22) and large pore zeolites (Y, Beta, Mordenite, SAPO-5, and ZSM-12) are evaluated for the transalkylation of alkyl aromatics to xylenes. However, challenges remain in optimizing zeolite catalysts for higher selectivity and conversion rates. The formation of undesirable by-products, such as polyaromatics, and the need for constant high reaction temperatures and pressures. Additionally, the limited stability of zeolites under harsh reaction conditions may reduce catalyst lifetime, requiring the development of more robust and selective catalysts for improving xylene yields. Hence, the development of an efficient catalyst and optimizing process parameters are challenges to obtaining higher selectivity and conversion.

Accordingly, one object of the present disclosure is to provide a catalyst composition for transalkylation of heavy reformates that may circumvent the drawbacks and limitations, such as, high pressure and temperature requirement, high operational economics, and formation of toxic byproducts, of methods and materials known in the art.

### SUMMARY

In an exemplary embodiment, a catalyst composition is described. The catalyst includes core-shell mesoporous silica-beta zeolite (BZ@MSi) composite particles including a mesoporous silica shell, a beta zeolite core particle, and nickel. The mesoporous silica shell encompasses the beta zeolite core particle. Further, the thickness of the mesoporous silica shell is in a range from 10 nanometer (nm) to 100 nm. The nickel is dispersed over the BZ@MSi composite particles, and the nickel is present in an amount ranging from 0.1 percent by weight (wt. %) to 10 wt. % relative to the total weight of the BZ@MSi composite particles.

In some embodiments, the thickness of the mesoporous silica shell is in a range from 20 nm to 60 nm.

In some embodiments, the thickness of the mesoporous silica shell is in a range from 30 nm to 50 nm.

In some embodiments, the thickness of the mesoporous silica shell is in a range from 35 nm to 45 nm.

In some embodiments, the nickel is present in an amount ranging from 0.5 wt. % to 3 wt. % relative to the total weight of the BZ@MSi composite particles.

In some embodiments, the nickel is present in an amount ranging from 0.75 wt. % to 1.25 wt. % relative to the total weight of the BZ@MSi composite particles.

In some embodiments, the silicon dioxide (SiO₂)/aluminium silicate (Al₂O₃) ratio of the beta zeolite core particles is in a range from 20 to 45.

In some embodiments, an SiO₂/Al₂O₃ ratio of the beta zeolite core particles is in a range from 23 to 28.

In some embodiments, the SiO₂/Al₂O₃ ratio of the beta zeolite core particles is in a range from 36 to 41.

In another exemplary embodiment, a method for the transalkylation of heavy aromatics is described. The method includes contacting a feed stream including a mixture of C₉₊ aromatics and toluene with a reactor bed including the catalyst described above. The method further includes transalkylating the C₉₊ aromatics and toluene (C₇H₈) to form a product stream enriched in xylene (C₆H₄(CH₃)₂) in comparison to the feed stream and collecting the product stream from the reactor bed.

In some embodiments, the thickness of the mesoporous silica shell is in a range from 20 nm to 60 nm.

In some embodiments, the thickness of the mesoporous silica shell is in a range from 30 nm to 50 nm.

In some embodiments, the nickel is present in an amount ranging from 0.5 wt. % to 3 wt. % relative to the total weight of the BZ@MSi composite particles.

In some embodiments, the method includes incorporation of nickel in an amount ranging from 0.75 wt. % to 1.25 wt. % relative to the total weight of the BZ@MSi composite particles.

In some embodiments, an SiO₂/Al₂O₃ ratio of the beta zeolite core particles is in a range from 20 to 45.

In some embodiments, the SiO₂/Al₂O₃ ratio of the beta zeolite core particles is in a range from 23 to 28.

In some embodiments, the SiO₂/Al₂O₃ ratio of the beta zeolite core particles is in a range from 36 to 41.

In some embodiments, the xylenes content of the product stream is greater than or equal to 19 wt. % relative to the total weight of the product stream.

In some embodiments, the xylenes content of the product stream is greater than or equal to 25 wt. % relative to the total weight of the product stream.

In some embodiments, the xylenes content of the product stream is greater than or equal to 30 wt. % relative to the total weight of the product stream.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic flow chart depicting a method of forming a mesoporous nanocomposite, according to certain embodiments.

FIG. 2A shows a N2 adsorption-desorption isotherms of pristine beta zeolite catalyst (B38), mesoporous core-shell composites of beta zeolites with varying shell thickness (B38-L, B38-M, B38-H), and nickel (Ni) modified beta zeolite (Ni/B38-M), according to certain embodiments.

FIG. 2B shows a N2 adsorption-desorption isotherms of pristine beta zeolite (B25), core-shell composite (B25-M), and Ni-modified beta zeolite (Ni/B25-M), according to certain embodiments.

FIG. 3A illustrates the pore size distribution pattern for the pristine B38, mesoporous core-shell composites B38-L, B38-M, B38-H, and Ni/B38-M, according to certain embodiments.

FIG. 3B illustrates the pore size distribution pattern for the pristine B25, core-shell composite B25-M, and Ni/B25-M, according to certain embodiments.

FIG. 4 shows X-ray diffraction (XRD) patterns for pristine B38, mesoporous core-shell composites B38-L, B38-M, B38-H, and Ni/B38-M, according to certain embodiments.

FIG. 5A depicts the ammonia-temperature programmed desorption (NH3-TPD) profile of pristine B38 and mesoporous core-shell composites prepared from B38, according to certain embodiments.

FIG. 5B depicts the NH3-TPD profile of pristine B25 and mesoporous core-shell composites derived from B25, according to certain embodiments.

FIG. 6A is a field emission scanning electron microscopy (FESEM) for pristine B38 at a scale of 1 μm, according to certain embodiments.

FIG. 6B is a FESEM for pristine B38 at a scale of 1 μm, according to certain embodiments.

FIG. 6C is a FESEM for core-shell composite (B38-M) at a scale of 1 μm, according to certain embodiments.

FIG. 6D is a FESEM for B38-M at a scale of 1 μm, according to certain embodiments.

FIG. 7A shows a FESEM image of pristine B38 at a scale of 1 μm, according to certain embodiments.

FIG. 7B shows an energy dispersive X-ray spectroscopy (EDX) image of pristine B38 at a scale of 1 μm, according to certain embodiments.

FIG. 7C shows a FESEM images for core-shell composites B38-L, B38-M, and B38-H at a scale of 1 μm, according to certain embodiments.

FIG. 7D shows EDX images for core-shell composites B38-L, B38-M, and B38-H at scale of 1 μm, according to certain embodiments.

FIG. 8A shows a FESEM image of pristine B25, according to certain embodiments.

FIG. 8B shows a FESEM image of core-shell composite Ni/B25-M, according to certain embodiments.

FIG. 8C shows a EDX elemental mapping image of core-shell composite Ni/B25-M, according to certain embodiments.

FIG. 8D shows a EDX image of core-shell composite Ni/B25-M, according to certain embodiments.

FIG. 8E shows a EDX image of core-shell composite Ni/B25-M, according to certain embodiments.

FIG. 8F shows a EDX image of core-shell composite Ni/B25-M, according to certain embodiments.

FIG. 9A shows a high-resolution transmission electron microscopy (HRTEM) image of pristine B38 at scale of 20 nm, according to certain embodiments.

FIG. 9B shows a HRTEM image of core-shell composite B38-L at a scale of 100 nm, according to certain embodiments.

FIG. 9C shows a HRTEM image of core-shell composite B38-M at a scale of 100 nm, according to certain embodiments.

FIG. 9D shows a HRTEM image of core-shell composite B38-H at a scale of 200 nm, according to certain embodiments.

FIG. 9E shows a HRTEM image of core-shell composite Ni/B25-M at a scale of 200 nm, according to certain embodiments.

FIG. 9F shows a HRTEM image of core-shell composite Ni/B25-M at a scale of 10 nm, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “core-shell” refers to a structured material composed of two distinct parts: a core, which forms the inner component, and a shell, which encapsulates or surrounds the core. The core typically serves as the primary functional element, providing mechanical strength, magnetic properties, or other intrinsic features, while the shell acts as a protective or functional layer, offering stability, enhanced compatibility, or tailored interactions with the surrounding environment. This unique configuration allows core-shell materials to exhibit a combination of properties from both components, making them versatile for various applications such as catalysis, drug delivery, energy storage, and coatings. The interaction between the core and shell, particularly at their interface, contributes to determining the material's overall performance and suitability for specific purposes.

As used herein, the term “zeolite” refers to a crystalline, microporous material composed primarily of aluminosilicates, which are frameworks of silica (SiO₂) and alumina (AlO₂) tetrahedra linked by oxygen atoms. Zeolites possess a highly ordered structure with uniform pores and channels of molecular dimensions, making them unique materials for a wide range of applications. These pores can selectively adsorb, separate, or catalyze reactions involving small molecules. Zeolites are commonly classified based on their structure, pore size, and chemical composition, and they may occur naturally or be synthesized for specific uses. Their high surface area, ion-exchange capacity, and thermal and chemical stability make zeolites valuable in catalysis, gas separation, water purification, and other industrial processes.

As used herein, the term “mesoporous” refers to a material characterized by the presence of pores with diameters in the range of 2 to 50 nanometers, as defined by the International Union of Pure and Applied Chemistry (IUPAC). Mesoporous materials possess an ordered or disordered network of interconnected pores within this size range, providing a high surface area and tunable pore structure.

As used herein, the term “total acidity” refers to the overall measure of the acidic sites present in a material, typically expressed in terms of their ability to donate protons (Brønsted acidity) or accept electron pairs (Lewis acidity). Total acidity is a property in catalytic materials that influences their reactivity, selectivity, and efficiency in acid-catalyzed reactions. It is commonly determined using techniques such as ammonia-temperature programmed desorption (NH₃-TPD), pyridine adsorption coupled with Fourier-transform infrared spectroscopy (FTIR), or titration methods. The term encompasses both the quantity and strength of the acidic sites, which can vary depending on the material's composition, structure, and preparation methods.

As used herein, the term, “transalkylation” refers to a chemical process in which an alkyl group is transferred from one aromatic compound to another. This reaction typically involves the re-distribution of alkyl groups between heavier polyalkylated aromatics (such as di- or tri-alkylated benzenes) and lighter aromatic compounds (such as benzene or toluene) in the presence of a catalyst.

As used herein, the term “heavy aromatics” refers to aromatic hydrocarbons that contain multiple alkyl substituents or larger alkyl groups, resulting in higher molecular weights and boiling points compared to lighter aromatic compounds like benzene, toluene, or xylenes. Examples include di- or tri-alkylated benzenes, ethylmethylbenzenes, and heavier polycyclic aromatic hydrocarbons. Due to their limited direct use, heavy aromatics are often subjected to upgrading processes, such as transalkylation or hydrodealkylation, to produce more valuable light aromatic products for industrial applications.

As used herein, the term “transalkylation of heavy aromatics” refers to a chemical process in which alkyl groups are redistributed between heavy aromatic compounds (such as polyalkylated benzenes) and lighter aromatic hydrocarbons (such as benzene or toluene) to produce more valuable monoalkylated aromatics. This reaction typically occurs in the presence of an acidic catalyst, such as zeolites or solid acids, under controlled temperature and pressure conditions. The process is widely used in the petrochemical industry to convert less valuable heavy aromatic by-products into commercially relevant compounds like toluene and xylenes, which serve as feedstocks for the production of chemicals, plastics, and fuels.

Aspects of this disclosure are directed to a nickel-impregnated beta zeolite-containing mesoporous silica core-shell catalyst for the transalkylation of heavy aromatics to xylene. The catalyst of the present disclosure exhibits a much higher conversion of the heavy aromatics to xylene than pristine zeolite, resulting in a high xylene yield and a reduction in higher aromatics composition.

According to a first aspect of the present disclosure, a catalyst composition is described. The catalyst includes a core-shell mesoporous silica-beta zeolite (BZ@MSi) particles, which include a mesoporous silica shell (also referred to a shell) and a beta zeolite core particle (also referred to as a core), where the mesoporous silica shell encompasses the beta zeolite core particle. The BZ@MSi particles are typically spherical, and have an average diameter ranging in size from 100 nm to 1.5 μm depending on the specific composition.

Beta zeolite is a complex intergrowth family whose desilication stability is lower than ZSM-5 and mordenite. In some embodiments, the SiO₂/Al₂O₃ ratio of the beta zeolite core particles ranges from 10 to 100, 20 to 45, 23 to 28, 36 to 42, 25 to 25, 30 to 45, 35 to 45, or 40 to 45. In preferred embodiments, the SiO₂/Al₂O₃ ratio of the beta zeolite core particles is 38 or 25. In some embodiments, the catalyst has SiO₂/Al₂O₃ ratio from 23 to 300.

In some embodiments, the thickness of the mesoporous silica shell ranges from 1 to 100, 10 to 100 nm, 20 to 60 nm, 30 to 50 nm, 20 to 100 nm, 30 to 100 nm, 40 to 100 nm, 50 to 100 nm, 60 to 100 nm, 70 to 100 nm, 80 to 100 nm, or 90 to 100 nm. In preferred embodiments, the shell has a thickness of about 24 nm-54 nm.

In addition, the catalyst further includes nickel or oxides thereof dispersed over the BZ@Msi composite particles. The nickel is present in an amount ranging from 0.1 to 30 wt. %, 0.1 to 20 wt. %, 0.1 to 10 wt. %, 0.5 to 3 wt %, 0.75 to 1.25 wt. %, 0.75 to 1.25 wt. %, 1 to 10 wt. %, 2 to 10 wt. %, 3 to 10 wt. %, 4 to 10 wt. %, 5 to 10 wt. %, 6 to 10 wt. %, 7 to 10 wt. %, 8 to 10 wt. %, 9 to 10 wt. % relative to the total weight of the BZ@Msi composite particles. In a preferred embodiment, the percentage of nickel is about 1 wt. % relative to the total weight of the BZ@Msi composite particles.

In some embodiments, in addition to the dispersion of nickel or oxides thereof in the catalyst, the catalyst may further include at least one oxide of a metal element from the group VB, VIB, VIIIB, and lanthanides. The metal element may be V, Cr, Mo, Fe, Pt, and Ce. In a preferred embodiment, no other metal elements other than nickel are present in the catalyst.

In some embodiments, the catalyst may have a Brunauer-Emmett-Teller (BET) surface area ranging from 100 to 800 m²/g, 330 to 700 m²/g, 350 to 700 m²/g, 400 to 700 m²/g, 450 to 700 m²/g, 500 to 700 m²/g, 550 to 700 m²/g, 600 to 700 m²/g, or 650 to 700 m²/g. In preferred embodiments, the surface area of the catalyst is about 587 m²/g.

In some embodiment, the catalyst may have a pore volume ranging from 0.1 to 1 cm³/g, 0.2 to 1 cm³/g, 0.3 to 1 cm³/g, 0.4 to 1 cm³/g, 0.5 to 1 cm³/g, 0.6 to 1 cm³/g, 0.7 to 1 cm³/g, 0.8 to 1 cm³/g, 0.9 to 1 cm³/g. In preferred embodiments, the pore volume of the catalysts is about 0.717 cm³/g.

In some embodiment, the catalyst may have a total acidity ranging from 0.1 to 1 NH3 mmol/g, 0.2 to 1 NH3 mmol/g, 0.3 to 1 NH3 mmol/g, 0.4 to 1 NH3 mmol/g, 0.5 to 1 NH3 mmol/g, 0.6 to 1 NH3 mmol/g, 0.7 to 1 NH3 mmol/g, 0.8 to 1 NH3 mmol/g, 0.9 to 1 NH3 mmol/g. In preferred embodiments, the catalyst has a total acidity of about 0.798 NH3 mmol/g.

According to a second aspect of the present disclosure, a method of producing the catalyst is described. The method includes the steps of 1) preparing a core-shell mesoporous silica-beta zeolite composite particles and 2) impregnating the core-shell mesoporous silica-beta zeolite composite particles with a nickel salt to obtain the catalyst.

Preparing a core-shell mesoporous silica-beta zeolite composite particles: The step includes providing a catalyst precursor, preferably a beta zeolite, and a silica source, and dissolving in an alkaline solution at an ambient temperature (20-37° C.), stirring, or both to yield a dissolved solution. In some embodiments, the beta zeolite has a molar ratio of SiO₂/Al₂O₃ of at least 20, preferably 20 to 45, preferably 23 to 28, and preferably 36 to 41.

The silica source may be in the form of amorphous silica, fumed silica, colloidal silica, tetraethyl orthosilicate (TEOS), sodium silicate, potassium silicate, lithium silicate, or the like. In a preferred embodiment, the silica source is TEOS.

The thickness of the silica over the catalyst precursor, beta zeolite, can be controlled by varying the TEOS/beta zeolite ratio. In an embodiment, the TEOS/beta zeolite ratio is in the range of 0.85 to 1.7, preferably about 1 to 1.5, preferably about 1.2 to 1.3, more preferably about 1.28.

The dissolving step, also called desilication, may be conducted in the presence of a surfactant, where the surfactant is often called a templating agent for the catalyst. In some embodiments, the surfactant is absent. For example, and not by way of limitation, the surfactant is a cationic surfactant. The cationic surfactant may include a quaternary ammonium compound. For example, and not by way of limitation, the quaternary ammonium cationic surfactant may be cetyltrimethyl ammonium bromide (CTAB). Optionally, other surfactants like tetraethylammonium hydroxide (TEAOH), triethylamine (TEA), tetramethylammonium hydroxide (TMAOH), hexadecylamine (HDA), polyethylene glycol (PEG), tetrapropylammonium hydroxide (TPAOH) and mixtures thereof may also be used. Various amounts of surfactant are contemplated for inclusion in the catalyst precursor. For example, the catalyst precursor may include 1 wt. % to 10 wt. % of the surfactant, preferably 1 wt. % to 5 wt. % of the surfactant, for example, CTAB.

The dissolution is carried out for 2-6 hours, preferably about 3-5 hours, preferably for 4 hours, under constant stirring, followed by processing via techniques like centrifugation, washing, and calcination to obtain the catalyst. In some embodiments, the calcining step may occur for 4 to 8 hours, preferably 8 hours, at a temperature of 400 to 600° C., preferably 500-600° C., preferably about 550° C. for about 8 hours to obtain the core-shell mesoporous silica-beta zeolite composite particles.

Impregnating the core-shell mesoporous silica-beta zeolite composite particles: The core-shell mesoporous silica-beta zeolite composite particles are impregnated with nickel prior to a calcining step by methods known in art to form the catalyst. The nickel is present in an amount of 0.5 to 3 wt. %, preferably 0.75 to 1.25 wt. %, relative to the total weight of the core-shell mesoporous silica-beta zeolite composite particles.

The catalyst is a transalkylation catalyst and is suitable for converting C₉₊ alkyl aromatic hydrocarbons to a product stream comprising benzene, toluene, and xylene, particularly to commercially valuable xylenes. The feed stream to the conversion process generally comprises alkyl aromatic hydrocarbons in the carbon number range C₉ to C₁₁₊ that may include, for example, such hydrocarbons as propylbenzenes, ethylmethylbenzenes, tetramethyl benzenes, ethyldimethylbenzenes, ethylbenzene, methylpropylbenzenes, and mixtures thereof. The heavy aromatics feed stream, characterized mainly by C₉₊ aromatics, permits effective transalkylation of light aromatics, such as benzene and toluene, with the heavier C₉₊ aromatics to yield additional C₈ aromatics, such as xylenes. The heavy aromatics stream may be derived from the same or different known refinery and petrochemical processes, and/or may be recycled from the separation of the product from transalkylation.

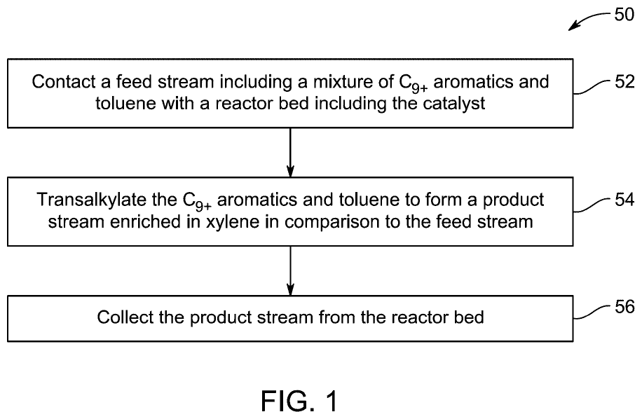

FIG. 1 illustrates a schematic flow chart of a method 50 for the transalkylation of heavy aromatics The order in which the method 50 is described is not intended to be construed as a limitation, and any number of the described method steps can be combined to implement the method 50. Additionally, individual steps may be removed or skipped from the method 50 without departing from the spirit and scope of the present disclosure.

At step 52, the method 50 includes contacting a feed stream including a mixture of C₉₊ aromatics and toluene with a reactor bed including the catalyst. In some embodiments, the mixture may include C₉₊ aromatics in an amount of 40-70 wt. %, preferably 45-65 wt. %, preferably 50-60 wt. %, preferably 55-60 wt. %, and more preferably about 60 wt. % in the mixture. In some embodiments, the amount of toluene present in the mixture is in the range of 30-60 wt. %, preferably 35-55 wt. %, preferably 40-50 wt. %, preferably about 40-45 wt. %, and more preferably about 40 wt. %. The C₉₊ aromatics feed used in this process will usually comprise one or more aromatic compounds containing at least 9 carbon atoms such as, e.g. trimethylbenzenes, dimethylbenzenes, and diethylbenzenes, etc. Specific C₉₊ aromatic compounds include mesitylene (1,3,5-trimethylbenzene), durene (1,2,4,5-tetramethylbenzene), hemimellitene (1,2,4-trimethylbenzene), pseudocumene (1,2,4-trimethylbenzene), 1,2-methylethylbenzene, 1,3-methylethylbenzene, 1,4-methylethylbenzene, propyl-substituted benzenes, butyl-substituted benzenes, isomers of dimethyl-ethylbenzenes, etc.

Suitable feed sources include a C₉₊ fraction of any refinery process rich in aromatics. This aromatics fraction contains a substantial proportion of C₉₊ aromatics, specifically C₉ to C₁₂ aromatic hydrocarbons. Typical refinery fractions that may be useful include catalytic reformate, FCC naphtha, or TCC naphtha.

In a preferred embodiment, the C₉₊ aromatics mainly include TMBs and MEBs. In some embodiments, the weight percentage of the TMBs in the mixture is around 35-45 wt. %, preferably 37-43 wt. %, preferably 38-40 wt. %, preferably 38-39 wt. %, more preferably about 38.3 wt. %. In some embodiments, the weight percentage of MEBs in the mixture is in the range of 5-20 wt. %, preferably 10-15 wt. %, preferably 11-14 wt. %, preferably 12-13 wt. %, preferably about 12.6 wt. %

In some embodiments, along with C₉₊ aromatics, the feed stream may further include numerous other hydrocarbons, preferably C₁₀₊ aromatics. Examples of such C₁₀₊ aromatics include, but are not limited to, butylbenzene, (including isobutylbenzene and tertiarybutylbenzene), diethylbenzene, methylpropylbenzene, dimethylethylbenzene, tetramethylbenzene, and C₁₁ aromatics, such as trimethylethylbenzene, and ethylpropylbenzene, for example. Examples of C₁₀₊ aromatics also can include naphthalene, and methylnaphthalene.

In some embodiments, the weight percentage of C₁₀ aromatics in the mixture is about 1-15 wt. %, preferably 2-13 wt. %, preferably 5-12 wt. %, preferably 6-10 wt. %, preferably about 6-8 wt. %, preferably about 6.4 wt. %.

In some embodiments, the feed stream includes 30-40 wt. % of toluene, 1-10 wt. % of xylene, 10-15 wt. % of MEBs, 35-45 wt. % of TMBs, 45-55 wt. % of C, aromatics, and 1-10 wt. % of C₁₀ aromatics. In a specific embodiment, the feed stream includes about 39.7 wt. % of toluene, 1.2 wt. % of xylene, 12.6 wt. % of MEBs, 38.3 wt. % of TMBs, 52.7 wt. % of C, aromatics, and 6.4 wt. % of C₁₀ aromatics.

In a specific embodiment, the feed mixture includes toluene, 1,2-dimethylbenzene, i-propylbenzene, n-propylbenzene, 1,3-methylethylbenzene, 1,4-methylethylbenzene, 1,3,5-trimethylbenzene, 1,2-methylethylbenzene, 1,2,4-trimethylbenzene, 1,2,3-trimethylbenzene, 1,4-methyl-1-propylbenzene, 1,3-diethylbenzene, 1,3-methyl-n-propylbenzene, 1,4-diethylbenzene, 1,4-dimethyl-2-ethylbenzene, 1,3-dimethyl-4-ethylbenzene, 1,2-dimethyl-4-ethylbenzene, 1,2-methyl-n-butylbenzene, and 1,2,4,5-tetramethylbenzene.

The reactor includes a single reactor with the catalyst or may include multiple reactors or stages. In one embodiment, the reactor has a fixed cylindrical catalyst bed; however, other reaction configurations utilizing moving beds of catalyst or radial-flow reactors or fluidized bed may be employed. Prior to the feed stream being delivered into the reactor bed, the catalyst may be reduced, for example, with a hydrogen gas for activation. In one embodiment, the catalyst is reduced with the hydrogen gas at 350-500° C., preferably at about 400-450° C., more preferably at about 400° C. for 5 hours.

In an embodiment, the feed stream is introduced into the reactor at a flow rate of 5-15 g/h, preferably 6-14 g/h, preferably 7-13 g/h, preferably 8-12 g/h, preferably 8-10 g/h, preferably 8-9 g/h, and more preferably about 8.4 g/h.

At step 54, the method 50 includes transalkylating the C₉₊ aromatics and toluene to form a product stream enriched in xylene in comparison to the feed stream. The transalkylation of the C₉₊ aromatics and toluene is preferably carried out in vapor phase in the reactor and in the presence of hydrogen. In one specific embodiment, the transalkylation is carried out at a temperature in the range of 250-500° C., preferably 300-450° C., preferably 325-400° C., preferably between 325-350° C. for 10-20 minutes, preferably for 12-18 minutes, preferably 14-16 minutes, and more preferably 15 minutes.

In some embodiments, the transalkylation reaction is performed at moderately elevated pressures of 1.0 MPa to 5.0 MPa. The liquid hourly space velocity (LHSV) is in the range of 1.0 hr⁻¹ to 10.0 hr⁻¹.

At step 56, the method 50 includes collecting the product stream from the reactor bed. In some embodiments, transalkylation of heavy aromatics may form products that include but are not limited to toluene, ethylbenzene, trimethylbenzenes, tetramethylbenzenes, mesitylene, dimethylbenzene, naphthalene, methylindane, dimethylnaphthalene, 1,2,4-trimethylbenzene, 1,3,5-trimethylbenzene, 1,2,3-trimethylbenzene, methylstyrene, ethylstyrene, methylnaphthalene, butylbenzene, pentylbenzene, hexylbenzene, heptylbenzene, octylbenzene, nonylbenzene, dodecylbenzene, phenylacetylene, biphenyl, fluorene, anthracene, phenanthrene, fluoranthene, pyrene, chrysene, dibenzothiophene, benzo (a) pyrene, dibenzofuran, tetrahydronaphthalene, indene, acetophenone, benzophenone, dihydroxybiphenyl, benzothiophene, naphthylmethane, and p-dimethylbenzene. In a preferred embodiment, transalkylation of heavy aromatics forms xylene as a product.

In some embodiment, the xylenes content of the product stream is greater than or equal to 19 wt. %, 25 wt. %, 30 wt. %, 20 wt. %, 24 wt. %, 26 wt. %, 28 wt. %, 30 wt. %, 32 wt. % relative to the total weight of the product stream. In a preferred embodiment, the xylenes content of the product stream is equal to 35 wt. % relative to the total weight of the product stream.

### Examples

The following examples demonstrate a catalyst composition including nickel modified mesoporous silica-beta zeolite (BZ@MSi) composite particles. The examples are provided solely for illustration and are not to be construed as limitations of the present disclosure, as many variations thereof are possible without departing from the spirit and scope of the present disclosure.

### Example 1: Experimental Procedure and Feed Composition

As listed in Table 1, the typical composition of the prepared feed was approximately 60 wt. % heavy reformate+40 wt. % toluene, used in the transalkylation reaction. Further, the combined reformate and toluene feed includes about 53 wt. % of C₉ aromatics, including mainly 38.3 wt. % TMBs and 12.6 wt. % MEBs. The composition further includes 6.4 wt. % of C₁₀ aromatics.

TABLE 1
Feedstock composition of the heavy reformate and toluene mixture.

| Retention | | |
|---|---|---|
| Time (min) | Component | wt. % |
| 7.53 | Toluene | 39.7 |
| 13.33 | 1,2-dimethylbenzene | 1.2 |
| 14.73 | i-propylbenzene | 0.4 |
| 15.81 | n-propylbenzene | 1.4 |
| 16.09 | 1,3-methylethylbenzene | 6.6 |
| 16.15 | 1,4-methylethylbenzene | 3.5 |
| 16.35 | 1,3,5-trimethylbenzene | 8.3 |
| 16.63 | 1,2-methylethylbenzene | 2.6 |
| 17.15 | 1,2,4-trimethylbenzene | 25.5 |
| 17.88 | 1,2,3-trimethylbenzene | 4.5 |
| 18.16 | 1,4-methyl-i-propylbenzene | 0.3 |
| 18.68 | 1,3-diethylbenzene | 0.4 |
| 18.78 | 1,3-methyl-n-propylbenzene | 0.3 |
| 18.85 | 1,4-diethylbenzene | 1 |
| 19.32 | 1,4-dimethyl-2-ethylbenzene | 0.5 |
| 19.36 | 1,3-dimethyl-4-ethylbenzene | 0.5 |
| 19.5 | 1,2-dimethyl-4-ethylbenzene | 1 |
| 20.24 | 1,2-methyl-n-butylbenzene | 1.1 |
| 20.32 | 1,2,4,5-tetramethylbenzene | 1.3 |
| Grouped | Toluene | 39.7 |
| Amounts | Xylenes | 1.2 |
| | MEBs | 12.6 |
| | TMBs | 38.3 |
| | C₉ aromatics | 52.7 |
| | C₁₀ aromatics | 6.4 |

### Example 2: Synthesis of Beta Core-Shell Composite

According to the present disclosure, the beta zeolites, with SiO₂/Al₂O₃ ratios of 25 and 38 were used as the reference zeolites core for the preparation of uniform mesoporous silica shell using a simple template-directed sol-gel approach, cetyltrimethylammonium bromide (CTAB) as template and tetraethyl orthosilicate (TEOS) were used as common silica precursor source.

Further, the preparation of core-shell composites, B38@MS-1, involves dispersing approximately 5 grams (g) of beta nanocrystals with a silica-to-alumina ratio (SiO₂/Al₂O₃) of 38 in a mixture of 3.5 g CTAB, 200 mL of water, 90 mL of ethanol (C₂H₆OH), and 13.7 mL of 25 wt. % ammonia (NH₃) solution. The B38 core serves as the microporous beta zeolite, while the mesoporous silica shell (MS) is formed through the sol-gel process to enhance the structural and catalytic properties. The obtained mixture is then stirred at ambient temperature for about 30 minutes. Afterwards, 4.25 g TEOS was added slowly, and the mixture was stirred for 4 hours at ambient temperature. The solid core-shell composites were isolated using a centrifugation technique at 4400 revolutions per minute (rpm), followed by washing with ethanol and water, and then dried overnight in the air at 80° C. The organic templates were removed by calcining at 550° C. for 8 hours in an air atmosphere to obtain B38@MS-1. The shell thickness was controlled by adjusting the TEOS/zeolite mass ratios between 0.85, 1.28, and 1.7. The prepared core-shell zeolite beta composites are listed in (Table 2).

TABLE 2
List of prepared core-shell zeolite beta composites.

| | | TEOS/Beta | Ni Loading |
|---|---|---|---|
| Beta Zeolite | SiO₂/Al₂O₃ | (wt. ratio) | (wt. %) |
| B38 | 38 | — | — |
| B25 | 25 | — | — |
| B38-L | 38 | 0.85 | — |
| B38-M | 38 | 1.28 | — |
| B38-H | 38 | 1.7 | — |
| B25-M | 25 | 1.28 | — |
| Ni/B38-M | 38 | 1.28 | 1 |
| Ni/B25-M | 25 | 1.28 | 1 |

### Example 3: Catalytic Evaluation

A fixed bed microflow reactor system, approximately 300 mm in length and 10.5 mm in inner diameter was used to convert heavy reformate and toluene. The setup for processing and reaction was designed to provide efficient and controlled flow conditions, which improved catalytic conversion. The reactor is heated using an electric furnace, confirming uniform temperature gradients. An internal thermocouple is installed to monitor the temperature of the catalyst bed. The product leaving the reactor was passed through a high-pressure gas-liquid separator. Further, it was cooled to 10° C. The gases exiting the separator pass through a back-pressure regulator to maintain the system pressure.

Typically, 2.0 g of the extruded catalyst 30-40 mesh size was loaded into the isothermal zone of the reactor, using 0.4 millimeter (mm) inert silicon carbide (SiC) placed at both the top and bottom of the catalyst bed for support. Prior to catalyst testing, the catalyst was activated in situ. The activation steps include drying under 100 milliliter per min (mL/min) nitrogen flow at 400° C. for 5 hours, followed by reduction under 100 mL/min flowing hydrogen at 450° C. for 5 hours. Afterwards, completing the catalyst activation, the reactor is cooled to 325° C., pressurized to 20 bars, and the liquid feed is introduced at a constant rate of 8.4 grams per hour (g/h). The reactor temperature was maintained at 325° C. for 15 minutes and was gradually increased to 350° C. at the rate of 1° C./min. A hydrogen flow of 100 mL/min is maintained throughout the evaluation. Liquid product samples were collected periodically during the steady-state operation and analyzed offline using a detailed hydrocarbon analyzer (DHA-PIONA) aligned gas chromatograph. The gas chromatograph disclosed a polydimethylsiloxane (PDMS-1) column with a length of 40 m and an inner diameter (ID) of 10 mm, enabling precise separation and analysis of hydrocarbon components.

### Example 4: Catalyst Characterization

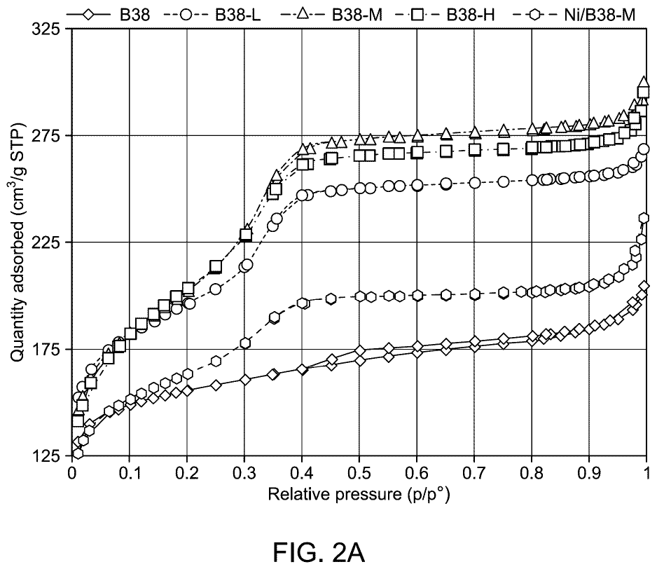

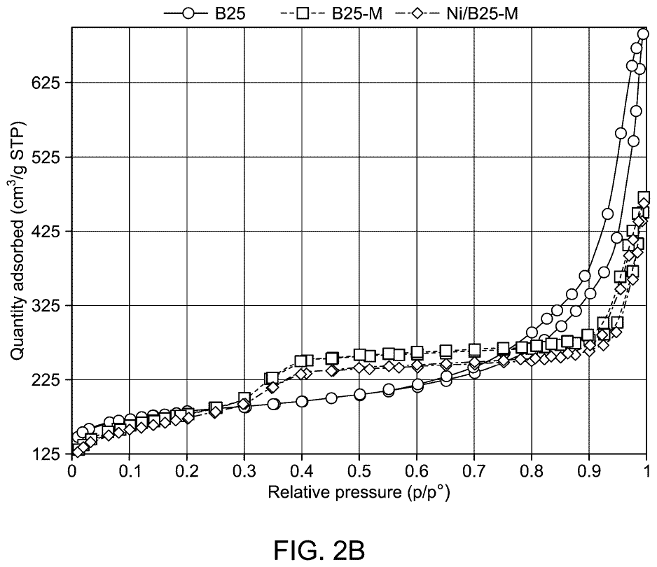

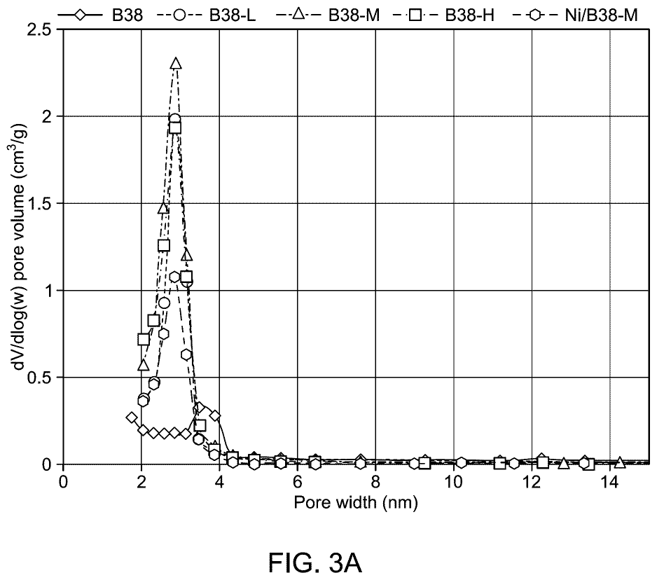

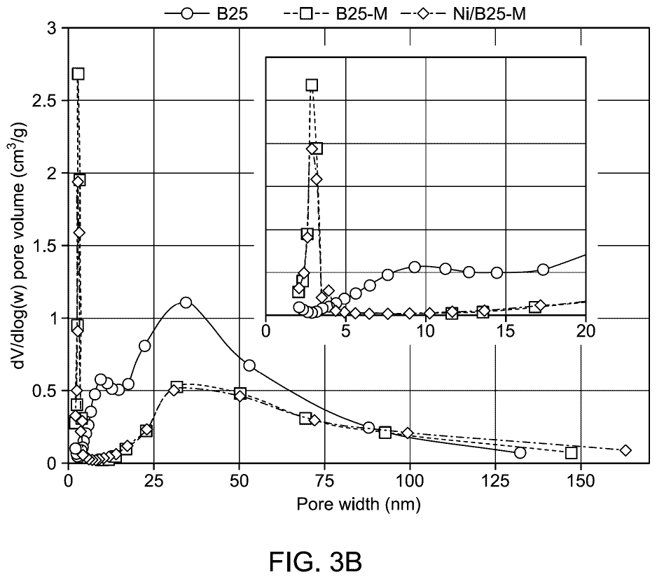

In the present disclosure, the textural properties of pristine beta zeolites (B38 and B25), beta zeolites including mesoporous silica core-shell composites (B38-L, B38-M, B38-H, and B25-M) and a pore size distribution (PSD) of the Ni-modified beta core-shell composite zeolites (Ni/B38-M and Ni/B25-M) were determined. Additionally, the PSD of the Ni-modified beta core-shell composite zeolites (Ni/B38-M and Ni/B25-M) were analyzed using N2 adsorption-desorption isotherms, as listed in Table 3. The adsorption-desorption isotherms of the pristine, mesoporous, and Ni-modified mesoporous zeolite beta catalysts with SiO₂/Al₂O₃ ratio of 38 and 25, as shown in FIGS. 2A-2B. The B38 catalyst exhibited type I isotherms with monolayer adsorption at a low relative pressure of P/P₀, as shown in FIG. 2A, which indicates the existence of a micropore framework, which is also clearly visible from the PSD curve, shown in FIGS. 3A-3B. Further, all mesoporous beta zeolites (B38-L, B38-M, B38-H, and Ni/B38-M) exhibited type IV isotherms with multi-layer adsorption, with the presence of a distinctive H4 hysteresis loop. The characteristic type IV isotherm with a sharp inflection observed at a relative pressure (P/P₀) of 0.3-0.4, attributed to capillary condensation in the mesopore range, which is also confirmed by the PSD curve, as shown in FIG. 3A. The pristine B25 exhibited a typical type IV isotherm with an H3 hysteresis loop, indicating the formation of slit-type mesoporous structure, as shown in FIG. 3B. Additionally, the B25-M and Ni/B25-M catalysts exhibited similar patterns as described for mesoporous catalysts based on B38. The PSD curves for all the catalysts are shown in FIGS. 3A-3B. A non-uniform distribution of pore sizes was apparent with an increase in pore volume, particularly noticeable in the case of B38. The B38 zeolite revealed pores within the 3-4 nm range, distinguished by its narrowed dimensions, while mesoporous B38 catalysts obtained pores ranging from 2-3 nm with deeper dimensions, as shown in FIG. 3A. The Ni/B38-M and Ni/B25-M catalysts experienced a reduction in desorption volume and pore size attributed to the impregnation of Ni, as shown in FIGS. 3A-3B. Furthermore, the impregnation led to the blocking of certain pores, which was confirmed by the decrease in both the total pore volume and mesoporous volume of the Ni-modified mesoporous beta zeolites, as listed in Table 3. A reduction in the relative crystallinity was observed from the XRD studies. Furthermore, Ni/B25-M particularly confirmed a bimodal pore size distribution, showcasing two different ranges of pore sizes, in contrast to other tested catalysts. The mesopore volume is increased as the Si/Al ratio decreases, which is due to more aluminum incorporated in the zeolite framework. Further, the structural defects and distortions in the zeolite framework resulted in additional void spaces and the formation of more mesopores, leading to an increase in mesopore area and volume.

Further, the surface area of B38 was observed using Brunauer-Emmett-Teller (BET) and found to be approximately 551 square meters per gram (m²/g). The surface area was increased as the coating of mesoporous silica increased, and the maximum value obtained for the B38-H catalyst was determined to be approximately 696 m²/g. The micropore surface area decreased and the mesopore surface area increased for mesoporous beta zeolites because of the increase in mesopore shell thickness. Furthermore, the mesoporous volume continued to increase as the mesoporous shell thickness increased over the surface of the B38 catalyst. However, with the impregnation of Ni on the B38-M catalyst, a decrease in both the specific surface area and mesopore surface area was noticed. The observed decline was generally attributed to pore blockage of NiO crystallites, causing the transformation of certain mesopores into micropores. Hence, the process leads to an increase in micropore surface area.

TABLE 3
Textural properties of pristine and mesoporous core-shell beta composites.

| Property | B38 | B38-L | B38-M | B38-H | Ni/B38-M | B25 | B25-M | Ni/B25-M |
|---|---|---|---|---|---|---|---|---|
| Surface area | | | | | | | | |
| BET Surface area | 551 | 644 | 695 | 696 | 536 | 462 | 607 | 587 |
| (m²/g) | | | | | | | | |
| t-Plot Micropore area | 386 | 319 | 236 | 204 | 264 | 287 | 235 | 228 |
| (m²/g) | | | | | | | | |
| t-Plot External | 165 | 325 | 458 | 492 | 272 | 175 | 373 | 359 |
| Surface area (m²/g) | | | | | | | | |
| Pore volume | | | | | | | | |
| Total Pore volume | 0.365 | 0.416 | 0.465 | 0.456 | 0.366 | 0.796 | 0.729 | 0.717 |
| (cm³/g) | | | | | | | | |
| t-Plot micropore | 0.203 | 0.162 | 0.114 | 0.101 | 0.134 | 0.151 | 0.116 | 0.113 |
| volume (cm³/g) | | | | | | | | |
| Mesopore volume | 0.162 | 0.254 | 0.351 | 0.355 | 0.232 | 0.645 | 0.613 | 0.604 |
| (cm³/g) | | | | | | | | |
| NH₃ TPD results | | | | | | | | |
| Total acidity (NH₃ | 0.939 | 0.83 | 0.849 | 0.593 | 0.77 | 0.822 | 0.818 | 0.798 |
| mmol/g) | | | | | | | | |
| Peak I (100° C.-350° C.) | 0.831 | 0.69 | 0.651 | 0.468 | 0.576 | 0.71 | 0.645 | 0.589 |
| Peak II (350° C.-550° C.) | 0.108 | 0.14 | 0.198 | 0.125 | 0.194 | 0.112 | 0.173 | 0.209 |
| Strong/Weak ratio | 0.13 | 0.203 | 0.304 | 0.267 | 0.337 | 0.156 | 0.268 | 0.355 |

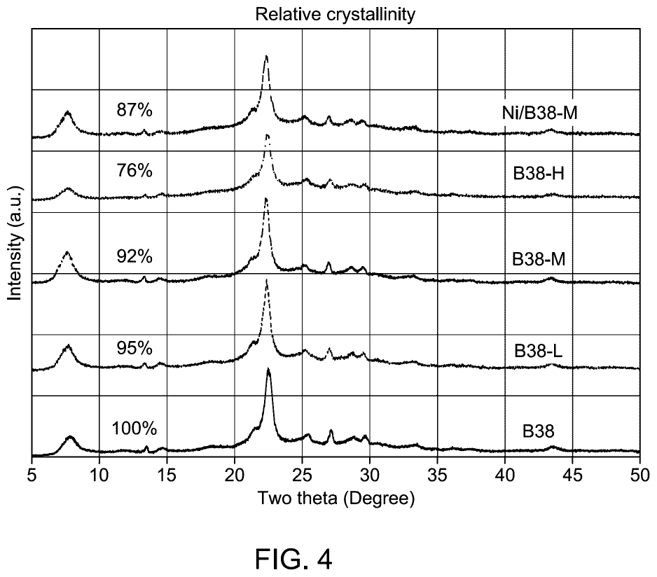

According to the present disclosure, X-ray diffraction (XRD) patterns were obtained for both pristine and core-shell composite catalysts. The characteristic diffraction peaks observed in both pristine and composite samples correspond to the beta zeolite topology, as shown in FIG. 4. The corresponding peaks were observed at 2θ values of 7.8°, 21.4°, 22.5°, 25.3°, 27.1°, 28.7°, 29.6°, and 43.2°. The beta zeolite structure remains stable for all core-shell beta zeolites after being treated with different amounts of silica (Si). All the catalysts exhibited high crystallinity. However, the relative crystallinity of the mesoporous B38 catalysts decreased as the mesoporous shell thickness increased. The relative crystallinity of the B38, B38-L, B38-M, and B38-H were about 100 wt. %, 95 wt. %, 92 wt. % and 76 wt. %, respectively. Further, the relative crystallinity of the Ni/B38-M catalyst decreased to 87 wt. % after the addition of the Ni on B38-M. However, the Ni-mesoporous beta zeolites prepared by the impregnation method exhibited diffraction peaks associated with the beta topology. However, no diffraction peaks corresponding to the Ni oxide species were observed, which indicates a high degree of dispersion.

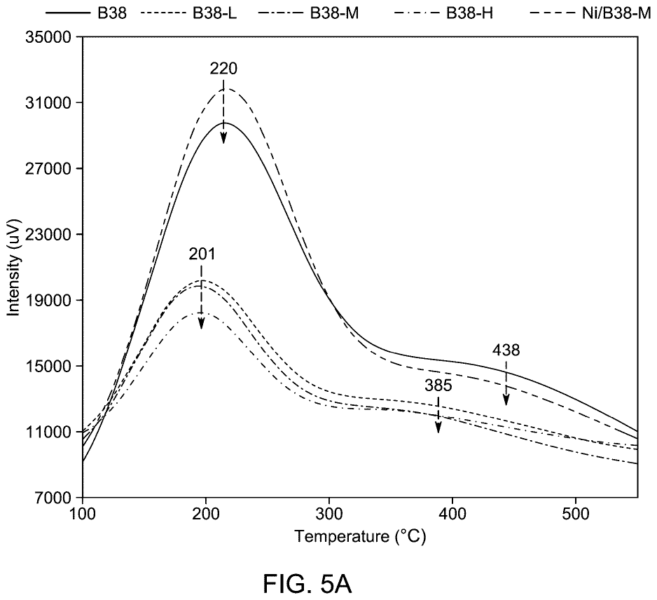

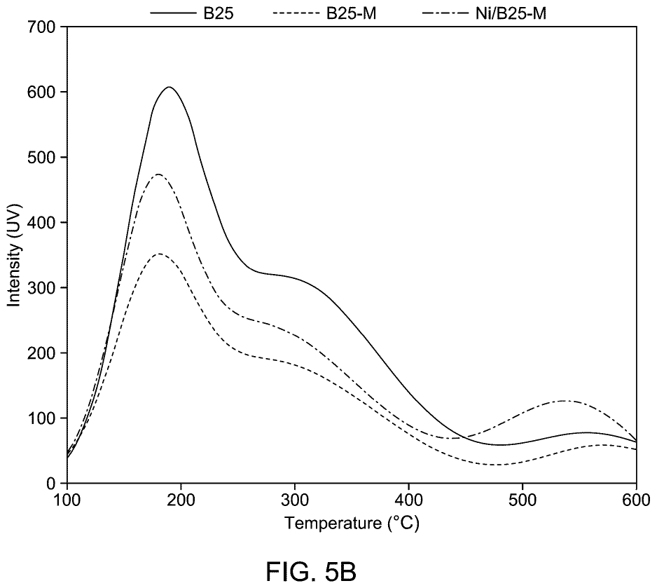

Further, ammonia temperature-programmed desorption (NH3-TPD) profiles of the pristine B38, mesoporous core-shell composites of beta zeolites of increased shell thickness (B38-L, B38-M, and B38-H), and Ni modified B38-M catalysts, as shown in FIG. 5A. The NH₃-TPD profiles of pristine B38 exhibited two different distinct peaks at desorption temperatures 220° C. and 438° C. The low temperature desorption peaks from 100° C. to 350° C. corresponds to weak-medium acid sites, while the high temperature desorption peak from 350° C. to 550° C. corresponds to strong acid sites. The NH₃-TPD profiles for core-shell composites shows two peaks at 201° C. and 385° C. with a left shift of about 19° C. and 53° C., respectively. The corresponding shift was primarily due to structural modifications around the pristine core of B38 by mesoporous silica. The total acid site concentration followed by B38>B38-M>Ni/B38-M>B38-L>B38-H, while the strong acidity of the catalysts was in the following order: B38-M approximately equal (~) to Ni/B38-M>B38-L>B38-H>B38, as listed in Table 2. Furthermore, the core-shell composite catalyst with medium thickness (B38-M) obtained higher total acidity than B38-L and B38-H. The Ni-modified core-shell zeolite beta catalyst showed some moderation in the strength and amount of weak acidity. However, the introduction of transition metal into the core-shell zeolite support are accountable for forming additional ammonia adsorption centers. A similar of behavior was observed in the earlier investigations when hierarchical composites of MCM-41 on zeolite beta were impregnated with Mo. The results obtained were noticeable for effect of incorporating 1 wt. % Ni from the higher ratio of strong/weak acid sites.

Further, NH3-TPD profiles of the B25, B25-M, and Ni/B25-M catalysts, as shown in FIG. 5B. Similar way, two different desorption peaks for weak-medium from 100° C. to 350° C. and strong acid sites from 350° C. to 550° C. were observed. The strength of acidity of the core-shell composites (B25-M and Ni/B25-M) decreased when compared to pristine B25 by shifting the peaks to lower temperatures in the weak-medium temperature region, as shown in FIG. 5B. The total acid site concentrations of the catalysts were in the following order: B25≈B25-M>Ni/B25-M. However, the strong acidity showed the trend: Ni/B25>B25-M>B25. The incorporation of Ni on B38-M and B25-M showed different effects on the total acidity. The total acidity of the Ni/B38-M increased after the incorporation of Ni onto B38-M. Conversely, the total acidity of the Ni/B25-M decreased after Ni impregnation on B25-M. Further, the lower Si/Al ratio of the B25-M initially has higher acid concentrations. Further, Ni incorporation, some of the acidic sites may be neutralized, block pores (pore volume and surface area decrease), or change the electronic properties in a way to decrease the total acidity. However, the strong acidity of Ni/B25-M catalysts increased after Ni incorporation and, hence, the strong/weak acidity ratio was highest for Ni/B25-M. Overall, the Ni/B25-M catalyst possesses higher stronger acidity compared to all other catalysts studied.

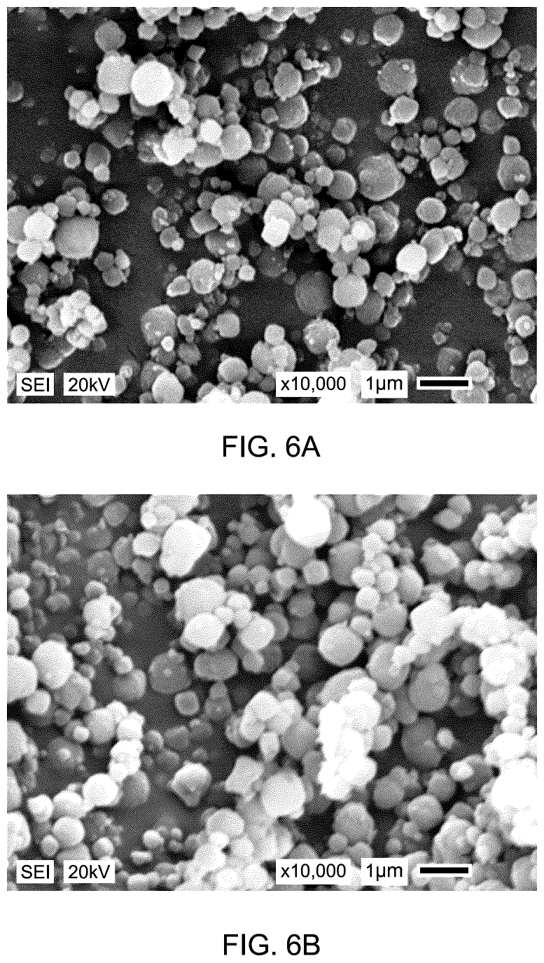

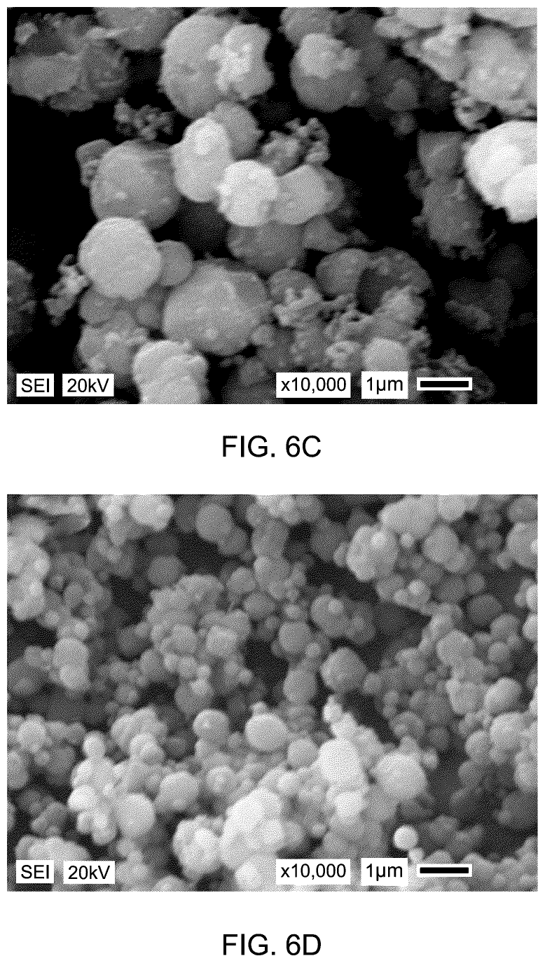

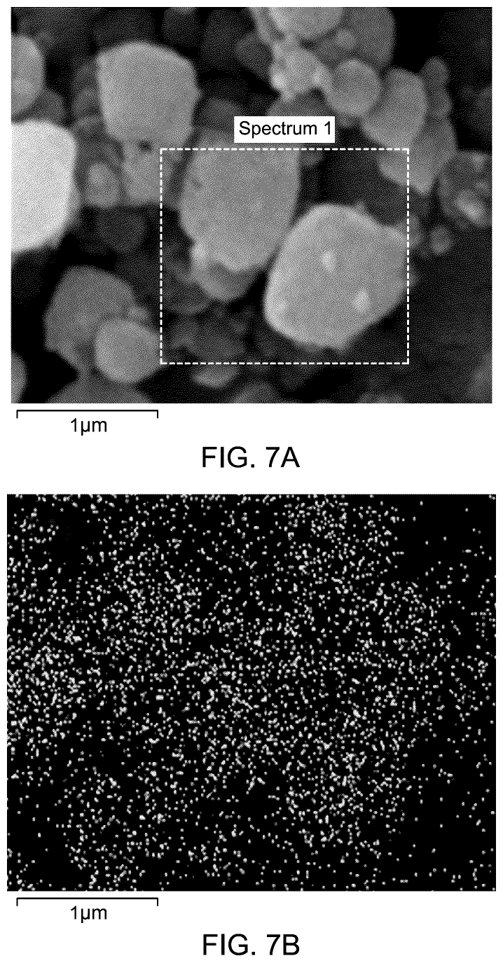

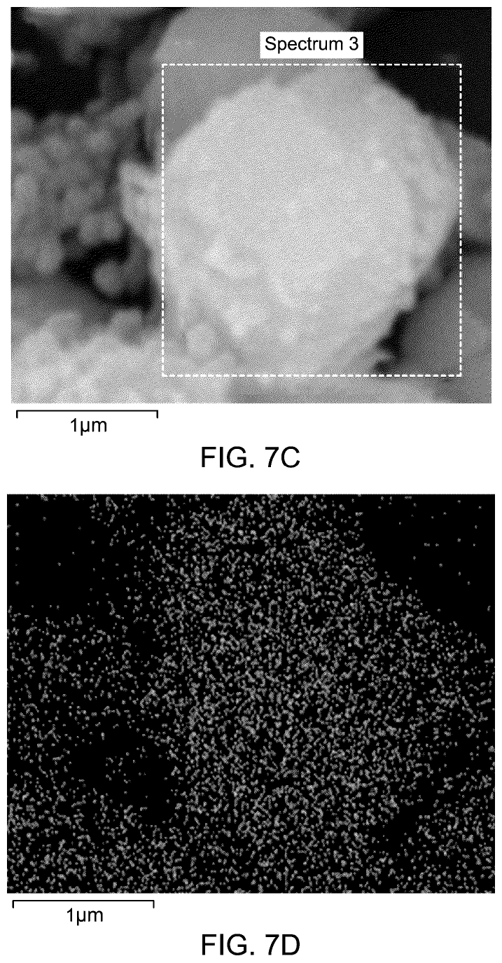

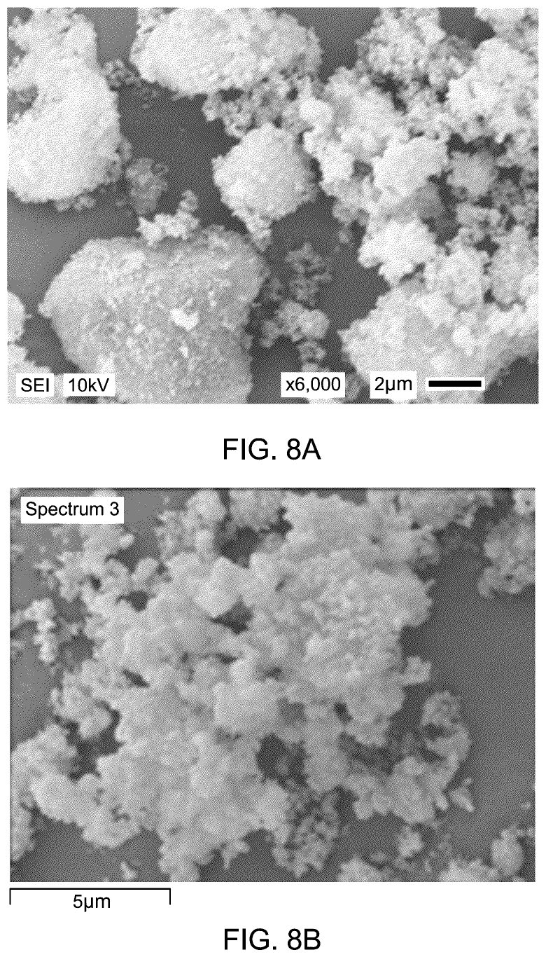

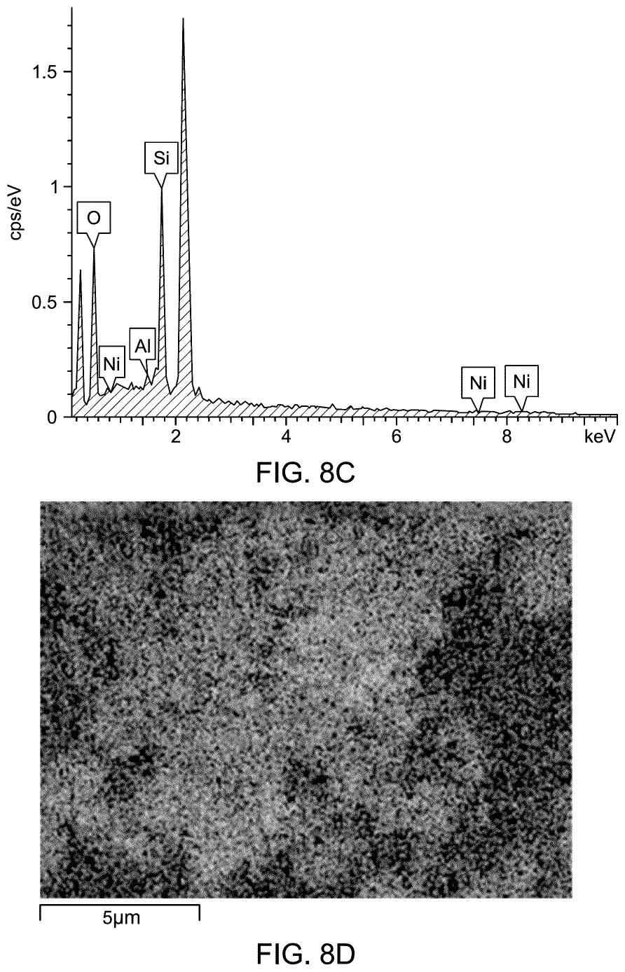

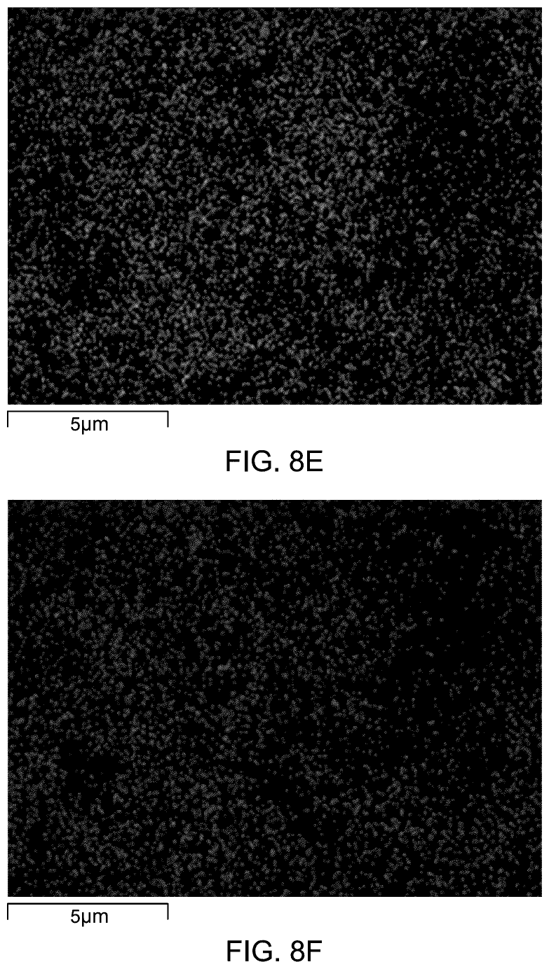

As can be seen from FIGS. 6A-6D, FESEM performed to evaluate the morphological characteristic of pristine zeolite beta and core-shell composites denoted as B38, B38-L, B38-M, and B38-H for elemental composition. The pristine zeolite beta exhibited well-ordered spherical shape crystallites, as shown in FIG. 6A. Further, the core-shell composites showed similar spherical crystallites and also exhibited the formation of another layer over the surface of the spherical crystallites, indicating that the shell was developing as the silica loading increased, as shown in FIGS. 6B-6D. Furthermore, FIGS. 7A-7D show SEM images and elementary mapping of the B38 and B38-M catalysts. Further, the EDS mapping analysis verified that the Si element was present in both samples, but the quantity drastically increased for core-shell zeolite indicating outer layer shell formation on the core, which is also confirmed by the FESEM analysis. The FESEM images and EDS mapping of the B25 and Ni/B25-M, shown in FIGS. 8A-8F. The enhanced mesoporous characteristics observed for Ni/B25-M catalyst is attributed to the structure of agglomerates formed by small spherical crystallites as revealed by FESEM. Energy dispersive X-ray analysis (EDX) revealed that Ni is present and homogeneously dispersed all over the catalyst surface, as shown in FIGS. 8C-8F.

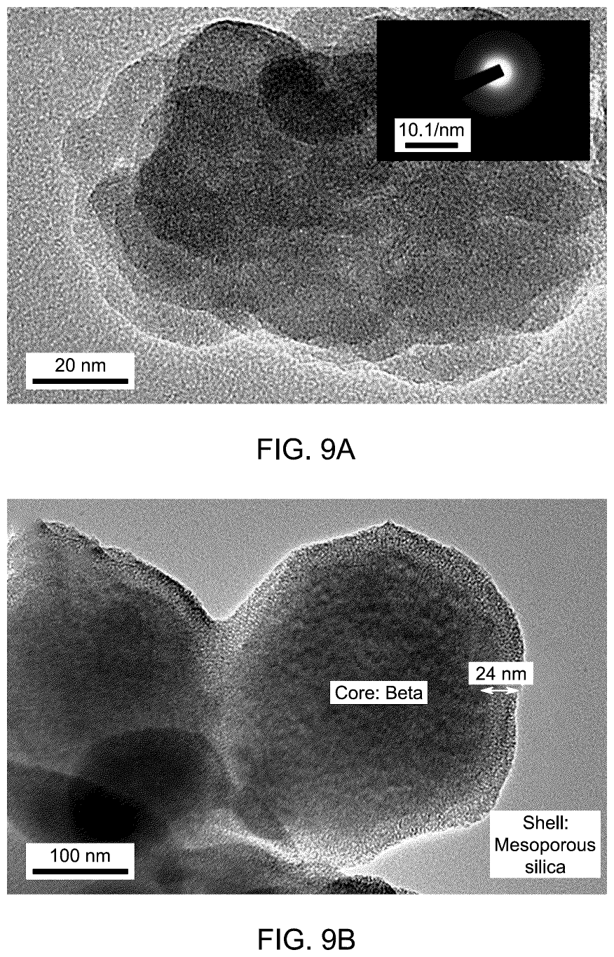

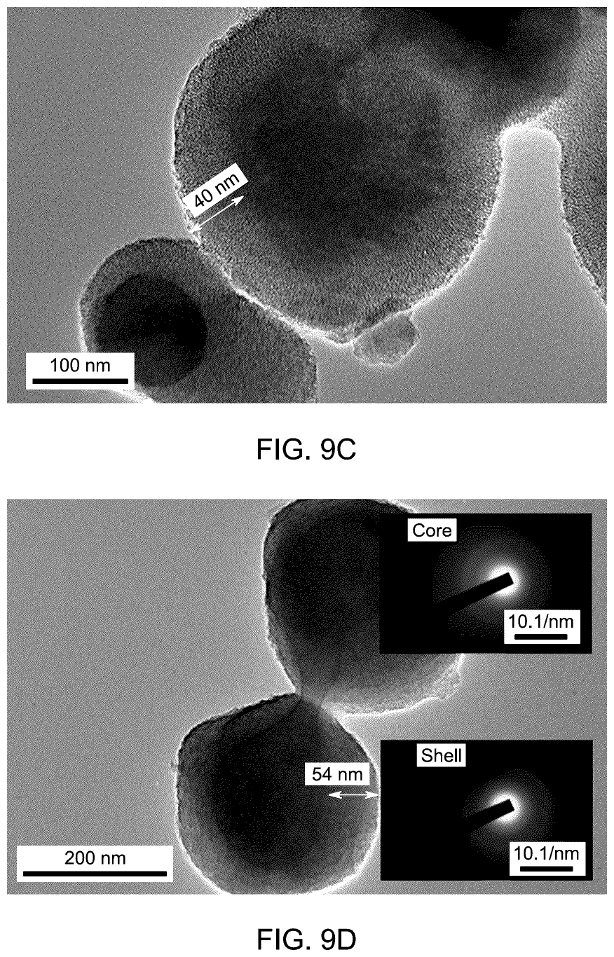

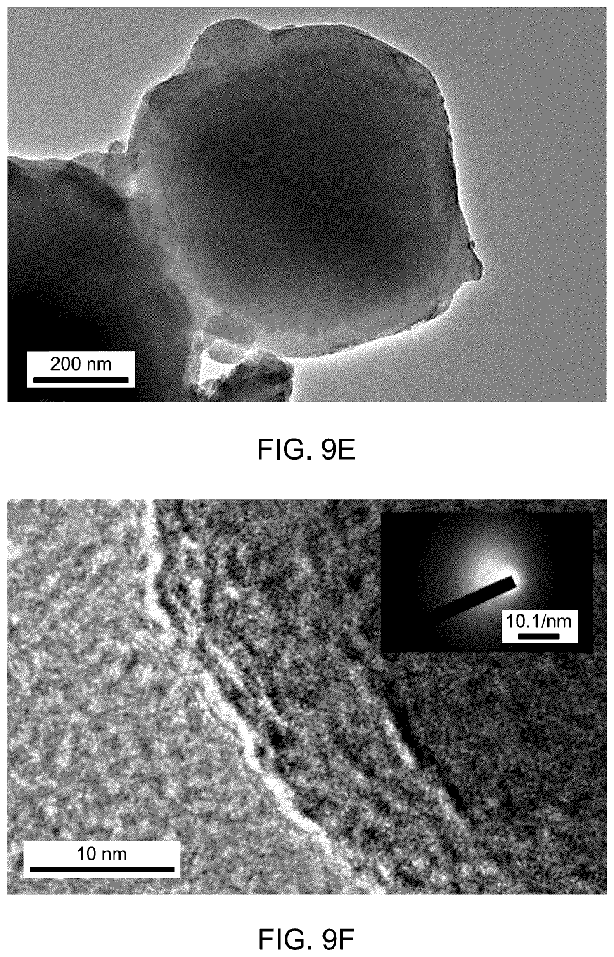

Further, the in-depth crystal shape, particle size, and Ni distribution were disclosed by the high-resolution transmission electron microscopy (HRTEM) images of pristine beta zeolite, and the modified mesoporous core-shell composites zeolite of varied shell thickness, (as shown in FIGS. 9A-9F). The results showed agglomeration of single crystals in the size range of approximately 19 nm to 25 nm for pristine B38 zeolite, (as shown in FIG. 9A). Further, the high-resolution images of core-shell composites provide evidence of the formation and enhancement of a shell over the B38 core as the shell thickness increased. The thickness of the shell formed over the core zeolite beta increased as the TEOS/B38 ratio increases from 0.85 to 1.28 to 1.70, resulting in the formation of core-shell composites B38-L, B38-M, and B38-H catalysts, respectively.

In the present disclosure, the multiple parallel and consecutive reactions take place during heavy reformate conversion due to the presence of several aromatic compounds. Dealkylation of MEBs is a reaction for the in-situ production of toluene. The amount of toluene determines the TMB-to-toluene ratio in the system, which regulates the kinetics and thermodynamics of consecutive reactions. The major transalkylation reaction between toluene and TMB involves the transfer of a methyl group from TMB to toluene to form xylenes. The transalkylation reaction is of paramount importance since it forms two moles of xylenes from a mole each of toluene and TMB. Disproportionation of TMB to form xylene and tetraethylmethylbenzenes (TeMBs) also occurs but it is thermodynamically limited. Isomerization of TMBs, MEBs, and xylenes occurs simultaneously with the other reactions. The isomers present at any time tend to shift toward the thermodynamic equilibrium ratio. The paring reaction of TeMBs peels or “pares” methyl groups from the ring of TeMBs and combines them into longer, preferentially branched side chains. Such branched side-chain alkyl groups are easier to dealkylate from the ring resulting in the formation of toluene and lower alkanes.

Further, the heavy aromatic mixture obtained from a refinery was mixed with 40 wt. % toluene and used as the feedstock for the transalkylation-dealkylation catalytic activity over pristine and modified mesoporous beta core-shell zeolite catalysts. As shown in (Table 4), the heavy aromatics feed includes a composition of 52.7 wt. % C9 aromatics (38.3 wt. % trimethylbenzenes and 12.6 wt. % methyl ethyl benzenes), 39.7 wt. % toluene, 1.2 wt. % xylenes, and 6.4 wt. % C₁₀ aromatics. The transalkylation of heavy aromatics with toluene was investigated using a fixed-bed reactor over different catalysts such as pristine beta zeolite B38, core-shell composite catalysts with different shell thickness, and Ni-modified core-shell composite catalysts. The reactions were conducted under different process parameters, including temperature and weight hourly space velocity (WHSV).

The effect of shell thickness on the core-shell composite catalysts were evaluated for the transalkylation of heavy aromatics to xylenes at 350° C. and a WHSV of 4.2 h⁻¹ under a 20 bar H2 pressure environment. The detailed product composition and conversion of various feed components, (as listed in Table 4) along with the changes in the conversion of MEBs and TMBs as well as in the yield of xylenes due to the changes in the shell thickness. The conversion of MEBs and TMBs over pristine B38 was 25.4 wt. % and 25.5 wt. %, respectively, with a xylene yield of 15.2 wt. %. The lower dealkylation and transalkylation activity exhibited by the B38 catalyst may be ascribed to its microporous nature, which prohibits alkyl aromatics from reaching the active sites. Further, the lack of mesoporous volume did not provide sufficient space for the formation of biphenylic intermediates formed during transalkylation of TMBs and toluene. The coating of B38 with mesoporous silica shell resulted in substantial increase in mesoporous volume and higher conversion levels of both the C9 aromatic components. The conversion of MEBs and TMBs reached a level of 44.4 wt. % and 45.8 wt. %, respectively, resulting in a xylene yield of about 25 wt. % over B38-M catalyst which has a medium sized coating of mesoporous silica shell on the surface of the B38. Improved performance of B38-M catalyst may also be attributed to elevated stronger acidity, (as listed in Table 4), which plays a role in promoting dealkylation and transalkylation reactions. Furthermore, the xylene selectivity increased from 1.49 to 2.21 indicating a reduction in the formation of undesired products. However, further increase in the thickness of the coating on B38-H resulted in lower activity and selectivity due to decrease in acidity and mesoporous volume. The variation in the conversion of isomers of MEBs over B38 and core-shell composite catalysts, (as listed in Table 4). The results show that 1-methyl-3-ethylbenzene has much lower conversion (15.7 wt. %) over B38 than other isomers. Its conversion increased over core-shell composites and reached 37.4 wt. % over B38-M but reduced again to 15.8 wt. % as the shell thickness is increased. Such a trend is also observed in the conversion of other MEB isomers with a lesser augmentation. The obtained results indicate that mesoporous nature of core-shell catalysts facilitate higher dealkylation of bulkier molecules. Furthermore, B38-M catalyst resulted in the formation of more C1-C4 gases (3.4 wt. %) and benzene (2.5 wt. %) but less C₁₀₊ aromatics (3.8 wt. %) compared to B38 and B38-L catalysts. The examined results also reflect higher dealkylation activity owing to higher acidity.

Further, the variation in the conversion of isomers of TMBs over B38 and core-shell composite catalysts, (listed in Table 4) is more notable. Among the isomers of TMBs, 1,2,3-TMB exhibited higher conversion than others, while 1,3,5-TMB is the least reactive. The trend was in line with the results obtained earlier over mordenite-based catalysts. The symmetric structure of 1,3,5-TMB forms a strong bonding of methyl groups with the benzene ring to form a very stable compound. The compound exhibited a higher kinetic diameter than the other isomers. The pristine B38 exhibited less than 3 wt. % conversion of 1,3,5-TMB. Its conversion increased over core-shell composites and reached 30.7 wt. % over B38-M but reduced again to 6.1 wt. % as the shell thickness increased. A similar trend is noticed in the conversion of other TMB isomers, but the effect of core-shell thickness is moderate. The sharp differences in the conversion of TMB isomers highlight the positive influence of mesoporous catalyst structure in transalkylation reactions.

Further, the overall conversion of TMBs and MEBs and xylenes selectivity was, therefore, highest over B38-M. The enhancement in overall performance may be attributed to the augmented mesoporous volume and higher mesopore surface area resulting from the forming of a silica shell around the B38 core zeolite. The mesoporous core-shell composites showed substantial improvement in the catalytic activity, both in terms of conversion and yield. The yield of xylenes was 25 wt. % over B38-M compared to only 15.2 wt. % obtained over B38. The enhancement in the formation of a shell on the core zeolite beta was also confirmed by transmission electron microscope (TEM) observations. Among all mesoporous core-shell composites, B38-H exhibited high mesoporous surface area and higher shell thickness, as shown by TEM results. In contrast, the B38-H catalyst displayed lower activity towards TMBs and MEBs conversion than the other two core-shell composite catalysts (i.e., B38-L and B38-M) despite having external surface area and shell thickness. The diminished activity of the B38-H catalyst may be ascribed to its lower total acidity (as listed in Table 4), which was not conducive to achieving higher catalytic activity. The B38-M catalyst is distinguished by optimal characteristics, featuring elevated strong acidity, surface area, and pore volume. Thus, higher conversion of MEB is feasible through optimization of the catalyst properties like acidity, surface area, and pore volume as the dealkylation is influenced by the kinetics. Further, thermodynamic equilibrium governs transalkylation, disproportionation, and isomerization, and the maximum conversion is determined by the thermodynamic equilibrium reached under reaction conditions. Transalkylation of TMBs and toluene was the major reaction to form xylenes. The transalkylation and hydro-dealkylation reactions are more pronounced over B38-M catalysts due to suitable pore structure and acidity. A further study was conducted for Ni-modified B38-M and B25-M catalysts to obtain a higher yield of xylenes and catalytic activity under different process parameters.

In the present disclosure, a hydrogenation function is introduced into the mesoporous core-shell catalyst by incorporating Ni metal to formulate bifunctional catalysts, which promote hydrogenation of coke precursors, dealkylation, saturation of cracking products, and other related products. Nickel is more commonly known for its high hydrogenation activity; and is very helpful in the hydrodealkylation of MEB and suppresses further re-alkylation of the alkenes to aromatics by saturating them in the hydrogen environment. The incorporation of nickel, however, reduced the pore volume, including the mesopore volume, and increased the strong/weak acidity ratio. The effect of Ni incorporation on the catalytic performance was evaluated by comparing the product distribution obtained over B38-M and Ni/B38-M catalysts. The product distribution is listed in Table 4. The conversion of all the isomers of MEBs dropped, and the overall conversion of MEBs was reduced from 44.4 wt. % to 39.2 wt. %, which caused a reduction of about 7 wt. % in the amount of toluene in the product. The conversion of the isomers of TMBs also dropped, and the overall conversion of TMBs was reduced from 45.8 wt. % to 40.2 wt. %. It was observed that the reduction in the conversion of 1,3,5-TMB was highest among the three isomers of TMBs due to the higher kinetic diameter and lower mesopore volume of the Ni/B38-M catalyst. Although the conversion of the main C9 aromatic components decreased, incorporating Ni into the B38-M catalyst increased the yield of xylenes from 25.0 wt. % to 27.8 wt. %. The increase in the yield of xylenes after Ni incorporation may be attributed to the optimum balance between weak acid sites and strong acid sites, which accelerates the transalkylation, disproportion, and hydrodealkylation reactions towards the formation of xylenes. A reduction in the yields of C1-C4 gases and benzene was observed, along with the increase in C₁₀ aromatics.

The examination of the effect of SiO₂/Al₂O₃ ratio in beta zeolite led to the preparation of the Ni/B25-M catalyst using beta zeolite with a SiO₂/Al₂O₃ ratio of 25. Further, performance of BZ catalyst is compared with Ni/B38-M catalyst which was prepared using zeolite beta having a SiO₂/Al₂O₃ ratio of 38. Except for the SiO₂/Al₂O₃ ratio, both Ni/B25-M and Ni/B38-M catalysts were prepared by the same procedure and same amount of Ni was loaded on them. Reduction of SiO₂/Al₂O₃ ratio of zeolite beta caused slight increase in total acidity with more strong acid sites. The product compositions are listed in Table 4. The change of SiO₂/Al₂O₃ ratio from 38 to 25 resulted in higher conversion of all the isomers of MEBs resulting in about 11 wt. % increase in MEBs conversion. Improved dealkylation is also observed by the formation of higher amounts of C1-C4 gases and benzene. Higher conversion of all the isomers of TMBs was also observed. The conversion of 1,3,5-TMB, which is a relatively stable isomer, increased by more than 10 wt. % due to the change in the SiO₂/Al₂O₃ ratio. The increased transalkylation of TMBs resulted in higher consumption of toluene resulting in formation of 35.1 wt. % of xylenes. Moreover, the xylenes selectivity increased from 2.17 to 2.50. Therefore, based on the results of catalyst performance, the Ni/B25-M catalyst provided a higher yield of xylenes and selectivity compared to the other catalysts explored. The Ni/B25-M catalyst has a greater mesopore volume and possesses higher strong acidity, which is more advantageous for the diffusion and conversion of bulky higher aromatics (C9+).

TABLE 4
Feedstock composition of the heavy reformate after mixing with toluene and product
distribution over different pristine and mesoporous core-shell catalysts

| | | | | | | Ni M-modified Core- | |
|---|---|---|---|---|---|---|---|
| | | | Core-Shell Composite | | | Shell Composite | |
| | Feed | B38 | B38-L | B38-M | B38-H | Ni/B38-M | Ni/B25-M |
| Composition (wt. %) | | | | | | | |
| C1-C4 gases | 0 | 2 | 2 | 3.4 | 2 | 2.4 | 3.2 |
| Benzene | 0 | 1.5 | 1.4 | 2.5 | 1.5 | 1.8 | 2.5 |
| Toluene | 39.7 | 36.6 | 33.2 | 36 | 32.3 | 28.9 | 24.6 |
| Ethylbenzene | 0 | 1.2 | 1.3 | 1.6 | 1.4 | 1.7 | 1.9 |
| i-propylbenzene | 0.4 | 0 | 0 | 0 | 0 | 0 | 0 |
| n-propylbenzene | 1.4 | 0 | 0 | 0 | 0 | 0 | 0 |
| 1,3-dimethylbenzene | 0 | 7.5 | 9.7 | 13 | 10.7 | 16.4 | 20.6 |
| 1,4-dimethylbenzene | 0 | 4.1 | 5.3 | 6.2 | 4.2 | 5.2 | 6.7 |
| 1,2-dimethylbenzene | 1.2 | 3.6 | 4.6 | 5.7 | 4.6 | 6.2 | 7.8 |
| 1,3-methylethylbenzene | 6.6 | 5.5 | 5.4 | 4.1 | 5.5 | 4.5 | 3.7 |
| 1,4-methylethylbenzene | 3.5 | 2.5 | 2.4 | 1.9 | 2.5 | 2.1 | 1.7 |
| 1,3,5-trimethylbenzene | 8.3 | 8.1 | 7.7 | 5.8 | 7.8 | 6.4 | 5.5 |
| 1,2-methylethylbenzene | 2.6 | 1.3 | 1.3 | 1 | 1.3 | 1.1 | 0.9 |
| 1,2,4-trimethylbenzene | 25.5 | 18 | 17 | 13.2 | 17.3 | 14.6 | 12.8 |
| 1,2,3-trimethylbenzene | 4.5 | 2.5 | 2.3 | 1.8 | 2.4 | 2 | 1.7 |
| 1,4-methyl-propylbenzene | 0.3 | 0 | 0 | 0 | 0 | 0 | 0 |
| 1,3-diethylbenzene | 0.4 | 0 | 0 | 0 | 0 | 0 | 0 |
| 1,4-diethylbenzene | 0.3 | 0 | 0 | 0 | 0 | 0 | 0 |
| 1,4-dimethyl-2- | 1 | 1.1 | 1.2 | 0.9 | 1.3 | 1.4 | 1.5 |
| ethylbenzene | | | | | | | |
| 1,3-dimethyl-4- | 0.5 | 0.5 | 0.6 | 0 | 0.6 | 0.6 | 0.7 |
| ethylbenzene | | | | | | | |
| 1,2-dimethyl-4- | 0.5 | 0.5 | 0.6 | 0 | 0.6 | 0.6 | 0.7 |
| ethylbenzene | | | | | | | |
| 1,2-methyl-n-butylbenzene | 1 | 0.8 | 1 | 0.7 | 1 | 1.1 | 1.1 |
| 1,2,4,5-tetramethylbenzene | 1.1 | 1.2 | 1.4 | 1 | 1.4 | 1.4 | 1.2 |
| Grouped Amounts (wt.) | | | | | | | |
| Xylenes | 1.2 | 15.2 | 19.6 | 25 | 19.5 | 27.8 | 35.1 |
| Methylethylbenzenes | 12.6 | 9.4 | 9.1 | 7 | 9.3 | 7.7 | 6.3 |
| (MEBs) | | | | | | | |
| Trimethylbenzenes (TMBs) | 38.3 | 28.5 | 27 | 20.7 | 27.4 | 22.9 | 20 |
| C9 aromatics | 52.7 | 37.9 | 36.1 | 27.7 | 36.8 | 30.6 | 26.3 |
| C10+ aromatics | 6.4 | 5.6 | 6.4 | 3.8 | 6.7 | 6.8 | 6.5 |
| Conversion (wt. %) | | | | | | | |
| 1,2-methylethylbenzene | | 47.8 | 50 | 60.7 | 48.8 | 57.9 | 65.4 |
| 1,3-methylethylbenzene | | 15.7 | 18.3 | 37.4 | 15.8 | 31.1 | 43.6 |
| 1,4-methylethylbenzene | | 27.4 | 29.6 | 45.9 | 27.7 | 40.6 | 51.2 |
| MEBs | | 25.4 | 27.9 | 44.4 | 25.8 | 39.2 | 50.1 |
| 1,2,3-trimethylbenzene | | 44.9 | 48.3 | 59.6 | 47.3 | 56.2 | 61.5 |
| 1,2,4-trimethylbenzene | | 29.5 | 33.2 | 48.4 | 32.3 | 42.9 | 49.8 |
| 1,3,5-trimethylbenzene | | 3 | 7.9 | 30.7 | 6.1 | 23.2 | 34 |
| TMBs | | 25.5 | 29.5 | 45.8 | 28.4 | 40.2 | 47.7 |
| Xylenes Selectivity (%) | | 1.5 | 1.8 | 2.2 | 1.7 | 2.2 | 2.5 |

According to present disclosure, a series of hierarchical beta zeolite-including mesoporous silica core-shell catalysts was synthesized using a simple template-directed sol-gel approach. The examinations evaluated the impact of varying shell thickness, addition of Ni metal, and the SiO₂/Al₂O₃ ratio of zeolite beta on the catalytic conversion of heavy reformate and toluene into xylenes. Detailed characterization of synthesized catalysts was carried out using several techniques such as N2 adsorption-desorption, XRD, NH3-TPD, FESEM, TEM, SEM-EDX, XPS and NMR. The HRTEM images show that the thickness of the shell formed over the core zeolite beta increased as the TEOS/zeolite ratio increases. An increase in mesoporous volume, a drop in relative crystallinity, and changes in the acid site concentration were observed with the increase in shell thickness. An increase in the yield and selectivity of xylenes was attained over the core-shell composite catalysts compared to the parent zeolite (B-38). The catalyst with medium shell thickness (B38-M) showed the highest improvement in catalytic performance. Introduction of a hydrogenation function by incorporation of 1 wt. % nickel metal (Ni/B38-M) resulted in further improvement in the xylene yield while maintaining the selectivity. Lowering the SiO₂/Al₂O₃ ratio from 38 to 25 of the zeolite beta in the metal incorporated catalyst with medium thickness (Ni/B25-M) raised the xylenes yield to 35 wt. % and further improved the xylenes selectivity. The improved performance may be attributed to the higher mesoporous volume and strong/weak ratio of acid site concentration. Therefore, the potential application of core-shell composite catalysts for converting heavy aromatics into xylenes has been effectively identified.

An aspect of the present disclosure provides a process for producing xylenes through the transalkylation of heavy aromatics includes using a mesoporous core-shell beta zeolite composite catalyst. The feed stream including a mixture of C₉₊ aromatics and toluene was processed using a catalyst prepared by a template-directed sol-gel method in an alkaline medium. The method further includes cetyltrimethylammonium bromide (CTAB) as the template and tetraethyl orthosilicate (TEOS) as the silica source to form a core-shell beta zeolite structure including mesoporous silica composites. Further, the feed is contacted with beta zeolite-based catalyst, including at least one metal oxide, to yield xylenes. Furthermore, the heavy reformate stream used in the process consists of a mixture of 40 wt. % to 60 wt. % heavy reformate and 60 wt. % to 40 wt. % toluene, including C₉₊ alkyl-aromatic hydrocarbons. Further, the catalyst highlights a microporous beta zeolite core coated with a mesoporous silica shell, with shell thicknesses ranging from 10 nm to 100 nm, controlled by varying the TEOS/beta zeolite ratio from 0.1 to 3. The catalyst further includes at least one metal oxide derived from elements in groups VB, VIB, VIIIB, or lanthanides, such as vanadium (V), chromium (Cr), molybdenum (Mo), iron (Fe), nickel (Ni), platinum (Pt), and cerium (Ce). Metal oxide content ranges from 0.1 wt. % to 20 wt. %, and the zeolite's SiO₂/Al₂O₃ ratio ranges from 23 to 300, with specific ratios of 38 and 25.

Furthermore, the process operates under conditions of 300° C. to 750° C., pressures from 0.1 MPa to 5 MPa, and a weight hourly space velocity (WHSV) of 1-10 h⁻¹, under favorable conditions, the conversion of C₉₊ alkyl-aromatic hydrocarbons exceeds 60 wt. %, achieving xylenes yields greater than 40 wt. % with a selectivity of over 80%.

Numerous modifications and variations of the present disclosure are possible in light of the above teachings. It is, therefore, to be understood that within the scope of the appended claims, the invention may be practiced otherwise than as specifically described herein.

1. A catalyst composition, comprising core-shell mesoporous silica-beta zeolite (BZ@MSi) composite particles comprising
a mesoporous silica shell,
a beta zeolite core particle, and
nickel;
wherein the mesoporous silica shell encompasses the beta zeolite core particle,
wherein the thickness of the mesoporous silica shell is in a range from 10 to 100 nm,
wherein the nickel is dispersed over the BZ@MSi composite particles and the nickel is present in an amount ranging from 0.1 to 10 wt. % relative to the total weight of the BZ@MSi composite particles.

2. The composition of claim 1, wherein the thickness of the mesoporous silica shell is in a range from 20 to 60 nm.

3. The composition of claim 2, wherein the thickness of the mesoporous silica shell is in a range from 30 to 50 nm.

4. The composition of claim 3, wherein the thickness of the mesoporous silica shell is in a range from 35 to 45 nm.

5. The composition of claim 1, wherein nickel is present in an amount ranging from 0.5 to 3 wt. % relative to the total weight of the BZ@MSi composite particles.

6. The composition of claim 5, wherein nickel is present in an amount ranging from 0.75 to 1.25 wt. % relative to the total weight of the BZ@MSi composite particles.

7. The composition of claim 1, wherein an SiO₂/Al₂O₃ ratio of the beta zeolite core particles is in a range from 20 to 45.

8. The composition of claim 7, wherein the SiO₂/Al₂O₃ ratio of the beta zeolite core particles is in a range from 23 to 28.

9. The composition of claim 8, wherein the SiO₂/Al₂O₃ ratio of the beta zeolite core particles is in a range from 36 to 41.

10. A method for the transalkylation of heavy aromatics, comprising:
contacting a feed stream comprising a mixture of C₉₊ aromatics and toluene with a reactor bed comprising the catalyst of claim 1,
transalkylating the C₉₊ aromatics and toluene to form a product stream enriched in xylene in comparison to the feed stream; and
collecting the product stream from the reactor bed.

11. The method of claim 10, wherein the thickness of the mesoporous silica shell is in a range from 20 to 60 nm.

12. The method of claim 11, wherein the thickness of the mesoporous silica shell is in a range from 30 to 50 nm.

13. The method of claim 10, wherein nickel is present in an amount ranging from 0.5 to 3 wt. % relative to the total weight of the BZ@MSi composite particles.

14. The method of claim 13, wherein the nickel is present in an amount ranging from 0.75 to 1.25 wt. % relative to the total weight of the BZ@MSi composite particles.

15. The method of claim 10, wherein an SiO₂/Al₂O₃ ratio of the beta zeolite core particles is in a range from 20 to 45.

16. The method of claim 15, wherein the SiO₂/Al₂O₃ ratio of the beta zeolite core particles is in a range from 23 to 28.

17. The method of claim 16, wherein the SiO₂/Al₂O₃ ratio of the beta zeolite core particles is in a range from 36 to 41.

18. The method of claim 10, wherein the xylenes content of the product stream is greater than or equal to 19 wt. % relative to the total weight of the product stream.

19. The method of claim 18, wherein the xylenes content of the product stream is greater than or equal to 25 wt. % relative to the total weight of the product stream.

20. The method of claim 19, wherein the xylenes content of the product stream is greater than or equal to 30 wt. % relative to the total weight of the product stream.
